(19) United States
(12) Patent Application Publication
(10) Pub. No.: US 2009/0129539 A1
(43) Pub. Date: May 21, 2009

(54) COMPUTED TOMOGRAPHY METHOD AND SYSTEM

(75) Inventors: Paul E. Licato, Wauwatosa, WI (US); Brian Thomsen, Milwaukee, WI (US); Jaynne Leverentz, Johnson Creek, WI (US); Boris Mudrik, Nesher (IL); Sardar Mal Gautham, Waukesha, WI (US); Bradley Gabrielse, Oostburg, WI (US); Yan Sorkin, Karmiel (IL)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/944,043

(22) Filed: Nov. 21, 2007

(51) Int. Cl. A61B 6/03

(52) U.S. Cl. 378/5

(57) ABSTRACT

A method of diagnostic imaging is disclosed herein. The method includes acquiring CT projection data at a first x-ray energy level and a second x-ray energy level. The method includes reconstructing a first image pair using a basis material decomposition algorithm and the CT projection data. The method includes displaying a first image from the first image pair. The method includes transforming the first image pair into a second image using image data from the first image pair. The method also includes displaying a second image. A CT system is also disclosed.

### BACKGROUND OF THE INVENTION

The subject matter disclosed herein relates to a computed tomography method and system.

Typically, in computed tomography (CT) imaging systems, an x-ray source emits a fan-shaped or a cone-shaped x-ray beam toward a subject or object, such as a patient or a piece of luggage, positioned on a support. The beam, after being attenuated by the subject, impinges upon a detector assembly. The intensity of the attenuated x-ray beam received at the detector assembly is typically dependent upon the attenuation of the x-ray beam by the subject.

In known third generation CT systems, the x-ray source and the detector assembly are rotated on a rotatable gantry portion around the object to be imaged so that a gantry angle at which the fan-shaped x-ray beam intersects the object constantly changes. The detector assembly is typically made of a plurality of detector modules. Data representing the intensity of the received x-ray beam at each of the detector elements is collected across a range of gantry angles. The data are ultimately processed to form an image.

Conventional CT systems emit an x-ray with a polychromatic spectrum. The x-ray attenuation of each material in the subject depends on the energy of the emitted x-ray. Due to this relationship, images acquired with a polychromatic x-ray beam suffer from beam hardening artifacts as is well known by those skilled in the art. CT projection data that is acquired with a monochromatic x-ray beam does not suffer from beam hardening artifacts.

If CT projection data is acquired at multiple x-ray energy levels, it is possible to create images largely free from beam hardening artifacts that look like they were acquired with a monochromatic x-ray beam. Additionally, the CT projection data that is acquired at multiple x-ray energy levels contains additional information about the subject or object being imaged that is not contained within a conventional CT image.

The problem is that the conventional method of generating and displaying images of multiple types is slow and inefficient.

### BRIEF DESCRIPTION OF THE INVENTION

The above-mentioned shortcomings, disadvantages and problems are addressed herein which will be understood by reading and understanding the following specification.

In an embodiment, a method of diagnostic image includes acquiring CT projection data at a first x-ray energy level and a second x-ray energy level. The method includes reconstructing a first image pair using a basis material decomposition algorithm and the CT projection data. The method includes displaying a first image from the first image pair. The method also includes transforming the first image pair into a second image using image data from the first image pair and displaying a second image.

In another embodiment, a method of diagnostic imaging includes acquiring CT projection data at a first x-ray energy level and a second x-ray energy level. The method includes reconstructing a first image pair using a basis material decomposition algorithm and the CT projection data. The method includes transforming the first image pair into a first image using image data from the first image pair. The method includes displaying the first image. The method includes transforming the first image pair into a second image using image data from the first image pair. The method also includes displaying the second image.

In another embodiment, a CT system includes a rotatable gantry portion, an x-ray source mounted on the rotatable gantry portion, a detector assembly mounted on the rotatable gantry portion and a controller in communication with the rotatable gantry portion. The controller is configured to acquire CT projection data at a first x-ray energy level and a second x-ray energy level. The controller is configured to reconstruct a first image pair using a basis material decomposition algorithm and the CT projection data. The controller is configured to display a first image from the first image pair. The controller is configured to transform the first image pair into a second image using image data from the first image pair and the controller is also configured to display a second image.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a CT system in accordance with an embodiment;

FIG. 2 is a schematic representation of images that may be reconstructed using CT projection data from multiple x-ray energy levels in accordance with an embodiment;

FIG. 3 is a flow chart illustrating a method in accordance with an embodiment; and

FIG. 4 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

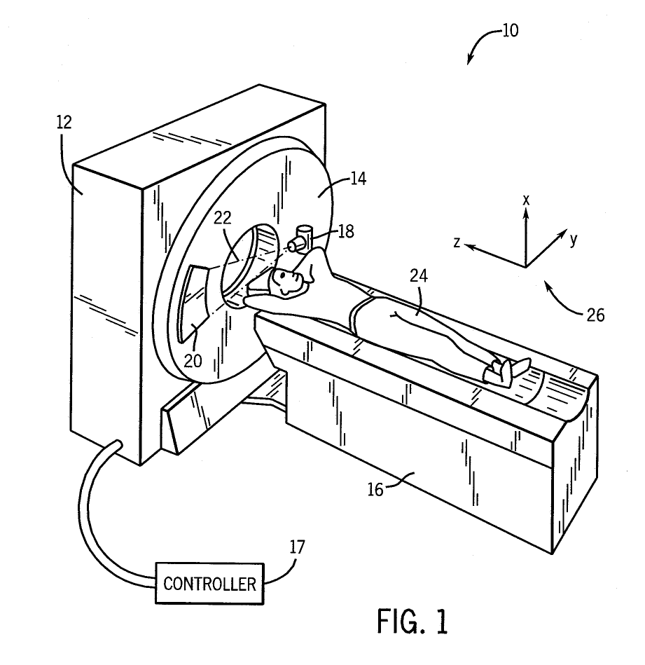

Referring to FIG. 1, a schematic representation of a computed tomography (CT) system 10 according to an embodiment is shown. The CT system 10 includes a gantry 12, a rotatable gantry portion 14, a support 16, and a controller 17. The rotatable gantry portion 14 is adapted to retain an x-ray source 18 and a detector assembly 20. The x-ray source 18 is configured to emit an x-ray beam 22 towards the detector assembly 20. The support 16 is configured to support a subject 24 being scanned. Hereinafter, the terms “subject” and “object” shall include anything capable of being imaged. The support 16 is capable of translating the subject 24 along a z-direction with respect to the gantry 12 as indicated by a coordinate axis 26. The controller 17 is in communication with the rotatable gantry portion 14. According to an embodiment, the controller 17 is electrically connected to the rotatable gantry portion. However, it should be understood that the controller could be in wireless communication with the rotatable gantry portion in other embodiments. The controller 17 may comprise software, firmware and/or hardware configured to control the acquisition of data, and the reconstruction and display of CT images. The controller may comprise subsystems (not shown) for controlling the on/off state of the x-ray source 18, a power level of the x-ray source 18, the position and rotational speed of the rotatable gantry portion 14, and the position of the subject 24 with respect to the gantry 12. The controller 17 may also be configured to control the transformation and reformatting of a reconstructed image as will be described in detail hereinafter.

The x-ray source 18 is configured to emit x-rays at multiple energy levels. For example, the x-ray source may emit x-rays at a first x-ray energy level and x-rays at a second x-ray energy level. The CT system 10 may be configured to acquire CT projection data through a variety of acquisition protocols. For example, acquisition protocols may include collecting CT projection data at the first x-ray energy level during a rotation of the rotatable gantry portion 14 and then collecting CT projection data at the second x-ray energy level during a separate rotation of the rotatable gantry portion 14. Or, the CT system 10 may be configured to acquired the CT projection data at multiple x-ray energy levels in an interleaved fashion. For example, the x-ray source 18 may be configured to switch between a first x-ray energy level and a second x-ray energy level multiple times during the rotation of the rotatable gantry portion 14. The CT system 10 may also be configured to acquire multiple x-ray energy level data at the same time. For example, this could include a detector assembly 20 that is configured to differentiate x-rays of a first x-ray energy level and x-rays of a second x-ray energy level. These three examples of ways that a CT system 10 could acquire CT projection data at multiple x-ray energy levels should be taken to be nonlimiting.

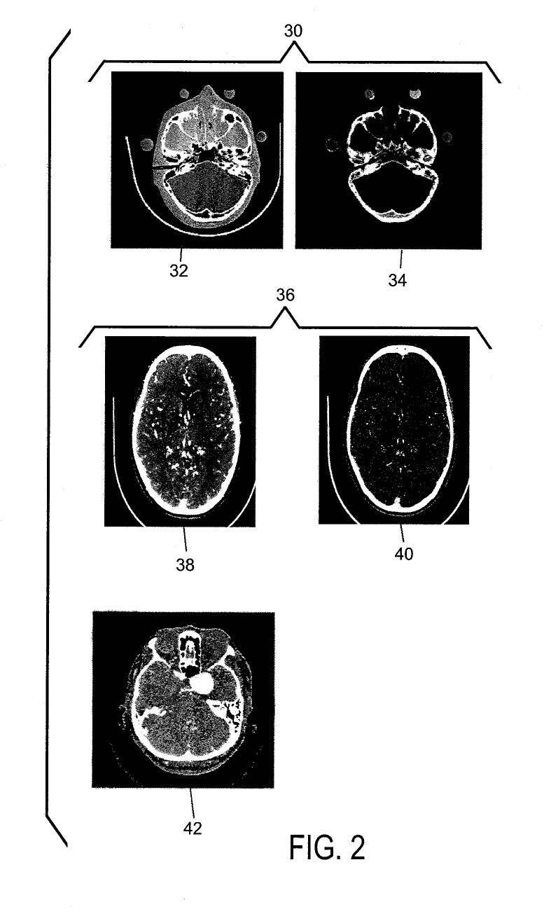

Referring to FIG. 2, schematic examples of images that may be reconstructed using CT projection data from multiple x-ray energy levels are shown in accordance with an embodiment. Hereinafter, an embodiment will be described that starts with a material density image pair 30 and transforms the material density image pair 30 into other types of images. It should be understood that it is not necessary to reconstruct the material density image pair 36 before reconstructing a monochromatic image pair 36. For example, according to another embodiment, it would be possible to start with the monochromatic image pair 36.

Once CT projection data has been acquired from multiple x-ray energy levels, the CT projection data may be used to reconstruct the material density image pair 30 using a basis material decomposition algorithm, which will be referred to as the BMD algorithm hereinafter. The BMD algorithm in accordance with an embodiment is described in detail in U.S. Pat. No. 6,904,118, which is hereby incorporated by reference in its entirety. The material density image pair 30 includes a material density image 32 with water as a basis material and a material density image 34 with iodine as a basis material. It should be understood that while water and iodine are used as exemplary basis materials, it would be possible to select any two basis materials for the BMD algorithm. According to an embodiment, the controller 17 (shown in FIG. 1) may selectively adjust the density of one or more of the basis materials. By selectively adjusting the densities of the basis materials, it is possible to adjust the density properties of a material density image created from the BMD algorithm as will be described in more detail hereinafter.

During the BMD algorithm, measured projections from the CT projection data are converted to a set of density line-integral projections. The density line-integral projections may be reconstructed to form a material density image pair 30 of each respective basis material. Once reconstructed, the material density images 32, 34 produced by the CT system 10 (shown in FIG. 1) reveal internal features of the subject 24 (shown in FIG. 1), expressed in the densities of the two basis materials. The material density image 32, 34 may be displayed to show these features. It should also be appreciated that while the BMD algorithm creates the material density image pair 30, it is not necessary to display both images of the material density image pair 30. In other words, it would be possible to only display one image of the material density image pair 30. It would also be possible to reconstruct the material density image pair 30 without displaying either of the images comprising the material density image pair 30.

According to an embodiment, the material density images 32, 34 created by the BMD algorithm may be transformed to produce the monochromatic image pair 36. The monochromatic image pair 36 includes a first monochromatic image 38 and a second monochromatic image 40. A monochromatic image is defined to include an image where the intensity values of the voxels are assigned as if a CT image were created by collecting projection data from the subject 24 (shown in FIG. 1) with a monochromatic x-ray beam. For the purposes of this disclosure, the monochromatic x-ray beam will be described as having a monochromatic x-ray energy level. Also, for the purposes of this disclosure, a monochromatic x-ray beam and an x-ray beam of a single x-ray energy level will be defined to be generally equivalent.

The first monochromatic image 38 schematically represents an image of a portion of the subject 24 (shown in FIG. 1) equivalent to that which would be created if the subject 24 had been exposed to an x-ray beam with a monochromatic x-ray energy level of 70 KeV. The second monochromatic image 40 schematically represents an image of the same portion of the subject 24 equivalent to that which would be created if the subject 24 had been exposed to an x-ray beam with a monochromatic x-ray energy level of 100 KeV. The intensities of the monochromatic image 38, 40 may be chosen to more clearly show a portion of the subject's 24 anatomy or to increase the contrast within a volume of interest. For example, the second monochromatic image 40 will contain information that is not present in the first monochromatic image 38 because the second monochromatic image 40 is equivalent to an image created with a higher energy x-ray beam. The monochromatic x-ray energy level may be chosen to increase the visibility of a volume of interest by optimizing qualities such as a contrast-to-noise ratio or a signal-to-noise ratio. Also, it should be understood that while the monochromatic image pair 36 is created, it would be possible to display only one of the monochromatic images 38, 40. According to an embodiment, the controller 17 (shown in FIG. 1) may be configured to selectively control the monochromatic x-ray energy level of the monochromatic image 38, 40. By selectively controlling the monochromatic x-ray energy level of the monochromatic images, it may be possible to quickly and easily display many different monochromatic images so that a user can objectively determine the most diagnostically useful monochromatic x-ray energy level.

The material density images 32, 34 created by the BMD algorithm may also be transformed to produce an effective atomic number image 42. The effective atomic number image 42 assigns voxel values based on the effective atomic number of the material being scanned rather than the x-ray attenuation as in a conventional CT image. As an example, any compound or mixture of materials measured with x-rays of multiple x-ray energy levels may be represented as a hypothetical material having the same x-ray energy attenuation characteristics. Unlike the atomic number of an element, an effective atomic number of a compound is defined by the x-ray attenuation characteristics and the concentration of the materials, and it need not be an integer. This effective atomic number representation property stems from a well-known fact that x-ray attenuation in the energy range useful for diagnostic x-ray imaging is strongly related to the electron density of compounds which is also related to the atomic number of materials. It should be understood that both the material density image pair 30 and the monochromatic image pair both contain enough data to be transformed into the effective atomic number image 42. For example, the effective atomic number image 42 may also be produced by transforming the monochromatic image pair 36

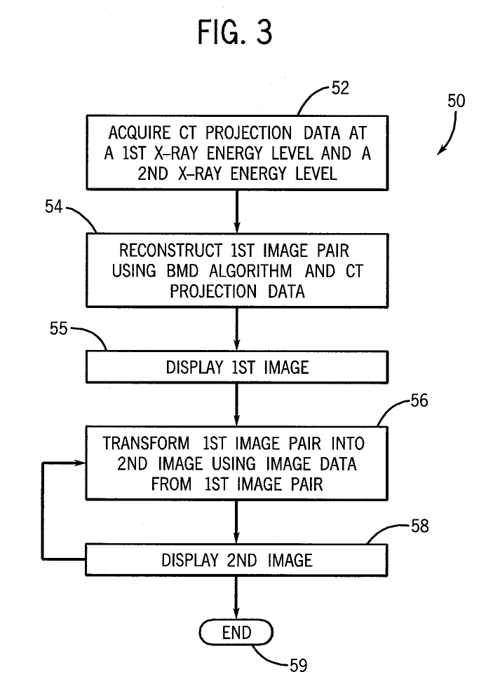

FIG. 3 is a flow chart representing a method 50 of diagnostic imaging in accordance with an embodiment. The individual blocks 52-59 of the method 50 represent steps that may be performed in accordance with method 50. The steps 52-59 need not be performed in the order shown.

Referring to FIG. 3, at step 52, CT projection data is acquired at a first x-ray energy level and at a second x-ray energy level. Acquiring the data at the first x-ray energy level and at the second x-ray energy level may take place according to any number of acquisition techniques including the techniques described in the discussion of CT system 10 (shown in FIG. 1).

At step 54, a first image pair is reconstructed using a BMD algorithm and the CT projection data acquired during step 52. An embodiment will be described where the first image pair is a material density image pair. However, it should be appreciated by those skilled in the art that the first image pair could comprise images of a different type in accordance with other embodiments. For the purposes of this disclosure, reconstructing a material density image pair includes assigning a value to each voxel of the image that correlates to the density of the subject at that particular three dimensional location, as is known by those skilled in the art. It should also be understood that reconstructing a material density image pair is distinct from displaying the material density image pair. At step 55, a first image from the first image pair is displayed.

At step 56, the material density image pair that was reconstructed at step 54 is transformed into a second image using image data from the material density image pair. For the purposes of this disclosure, the term transformed includes changing from an image type to another image type without acquiring new CT projection data. Transforming an image in the manner described in this disclosure is based on image data contained in the material density pair reconstructed at step 54. Transforming an image may be advantageous because it can potentially be many times faster than reacquiring another set of CT projection data or reconstructing another image. A nonlimiting list of possible transformations includes: transforming from a material density image pair into a monochromatic image pair; transforming from a material density image pair into a material density image pair with different basis materials; transforming from a monochromatic image pair into a material density image pair; transforming from monochromatic image pair into a monochromatic image pair showing one or more different x-ray energy levels; transforming from a material density image pair into an effective atomic number image; and transforming from a monochromatic image pair into an effective atomic number image. The transformations and image types were discussed in more detail in the previous discussion of FIG. 2.

At step 58 an image from the second image pair that was created during the transformation at step 56 is displayed. Displaying includes showing the image on a monitor, display, or on a printed document. Since the second image was created by transforming the first image pair at step 56, the first image can be ready for display very quickly. According to an embodiment, a user could selectively choose to transform the first image pair into a different image in generally real time. For the purposes of this disclosure, the term “generally real time” is defined to include a process that takes less than five seconds to complete. However, depending upon the specifics of the CT system 10 (shown in FIG. 1), the transformation from the image pair into a different image could occur in less than one second according to an embodiment. Since an embodiment shows transformed images in generally real time, it may be possible for the user to rapidly view many different transformed images. At step 58, if it is decided to view another image, the method 50 cycles back to step 56 where the first image pair can be transformed into another image. The method 50 may iteratively cycle through steps 56 and 58 as many times as are needed. Alternatively, if no additional transformed images are desired to be viewed, the method 50 advances to step 59 where it ends.

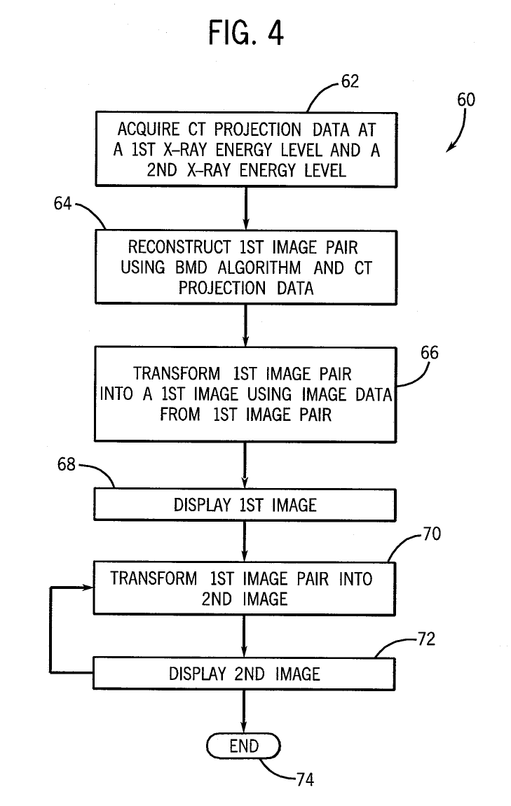

FIG. 4 is a flow chart representing a method 60 of diagnostic imaging in accordance with another embodiment. The individual blocks 62-74 of the method 60 represent steps that may be performed in accordance with method 60. The steps 62-74 need not be performed in the order shown.

Referring to FIG. 4, at step 62, CT projection data is acquired at a first x-ray energy level and at a second x-ray energy level. Acquiring the data at the first x-ray energy level and at the second x-ray energy level may take place according to any number of acquisition techniques including the techniques described in the discussion of CT system 10 (shown in FIG. 1).

At step 64, a first image pair is reconstructed using a BMD algorithm and the CT projection data acquired during step 62. For the purposes of this disclosure, reconstructing a material density image pair includes assigning a value to each voxel of the image that correlates to the density of the subject at that particular three dimensional location, as is known by those skilled in the art. It should also be understood that reconstructing a material density image pair is distinct from displaying the material density image pair.

At step 66, the first image pair that was reconstructed at step 64 is transformed into a first image using image data from the first image pair reconstructed at step 64. At step 68, the first image is displayed. At step 70, the first image pair is transformed into a second image. According to an embodiment, the second is different than the first image. At step 72, the second image that was created at step 70 is displayed. If an additional image required, the method 60 cycles back to step 70 where the an additional transformation of the first image pair occurs. If, at step 72, no additional transformed images are required, the method 60 ends at step 74.

Referring to both FIGS. 3 and 4, steps 55, 58, 68, and 72 each involve displaying an image. According to an embodiment, the image displayed at steps 55, 58, 68, and 72 may be displayed as a volume rendered image. Volume rendered images are three-dimensional representations that are well-known by those skilled in the art, so they will not be described in detail. Additionally, embodiments may manipulate the images in ways that are standard for visualizing computed tomography images. A non-limiting list of manipulations includes adjusting the contrast, brightness, window width, window level, and reformatting.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of diagnostic imaging comprising:
acquiring CT projection data at a first x-ray energy level and a second x-ray energy level;
reconstructing a first image pair using a basis material decomposition algorithm and the CT projection data;
displaying a first image from the first image pair;
transforming the first image pair into a second image using image data from the first image pair; and
displaying a second image.

2. The method of claim 1, wherein said reconstructing the first image pair comprises reconstructing a material density image pair.

3. The method of claim 2, wherein said displaying the second image comprises displaying a monochromatic image.

4. The method of claim 2, wherein said displaying the second image comprises displaying an effective atomic number image.

5. The method of claim 1, wherein said reconstructing the first image pair comprises reconstructing a monochromatic image pair.

6. The method of claim 5, wherein said displaying the second image comprises displaying a material density image.

7. The method of claim 5, wherein said displaying the second image comprises displaying an effective atomic number image.

8. A method of diagnostic imaging comprising:
acquiring CT projection data at a first x-ray energy level and a second x-ray energy level;
reconstructing a first image pair using a basis material decomposition algorithm and the CT projection data;
transforming the first image pair into a first image using image data from the first image pair;
displaying the first image;
transforming the first image pair into a second image using image data from the first image pair; and
displaying the second image.

9. The method of claim 8, wherein said transforming the first image pair into a first image comprises transforming the first image pair into an effective atomic number image.

10. The method of claim 9, wherein said transforming the first image pair into a second image comprises transforming the first image pair into either a material density image or a monochromatic image.

11. A CT system comprising:
a rotatable gantry portion;
an x-ray source mounted on the rotatable gantry portion;
a detector assembly mounted on the rotatable gantry portion; and
a controller in communication with the rotatable gantry portion, wherein the controller is configured to:
acquire CT projection data at a first x-ray energy level and a second x-ray energy level;
reconstruct a first image pair using a basis material decomposition algorithm and the CT projection data;
display a first image from the first image pair;
transform the first image pair into a second image using image data from the first image pair; and
display the second image.

12. The CT system of claim 11, wherein said controller configured to display the first image from the first image pair comprises a controller configured to display a material density image.

13. The CT system of claim 12, where said controller is further configured to selectively adjust a density of the material density image.

14. The CT system of claim 11, wherein said controller configured to display the first image from the first image pair comprises a controller configured to display a monochromatic image.

15. The CT system of claim 14, wherein said controller is further configured to selectively adjust a monochromatic x-ray energy level of the monochromatic image.

16. The CT system of claim 11, wherein said controller configured to display the second image comprises a controller configured to display a material density image.

17. The CT system of claim 11, wherein said controller configured to display the second image comprises a controller configured to display an monochromatic image.

18. The CT system of claim 11, wherein said controller configured to display the second image comprises a controller configured to display an effective atomic number image.

19. The CT system of claim 11, wherein said controller is further configured to display either the first image or the second image as a volume rendered image.

20. The CT system of claim 13, wherein said controller is further configured to selectively transition between the first image and the second image in generally real time.
